(19) United States
(12) Patent Application Publication
(10) Pub. No.: US 2024/0180789 A1
(43) Pub. Date: Jun. 6, 2024

(54) POLYMERIZATION INITIATOR-CONTAINING COMPOSITION AND DENTAL POLYMERIZABLE COMPOSITION

(71) Applicant: SUN MEDICAL CO., LTD., Shiga (JP)

(72) Inventors: Chidzuru INAMI, Moriyama-shi, Shiga (JP); Yoshihisa KAMIMOTO, Moriyama-shi, Shiga (JP); Kazuhiko YOSHINAGA, Moriyama-shi, Shiga (JP)

(21) Appl. No.: 18/280,708

(22) PCT Filed: Mar. 17, 2022
(86) PCT No.: PCT/JP2022/012212

§ 371 Date: Sep. 7, 2023

(30) Foreign Application Priority Data
Mar. 19, 2021 (JP) 2021-046656

(51) Int. Cl. A61K 6/79; A61K 6/60; C08F 120/14; C08K 5/18; C08K 5/21

(52) U.S. Cl. CPC A61K 6/79; A61K 6/60; C08F 120/14; C08K 5/18; C08K 5/21

(57) ABSTRACT

A polymerization initiator-containing composition contains a fourth period transition metal compound including a transition metal atom belonging to the fourth period of the Periodic Table, a tertiary amine compound, and a thiourea compound represented by the following general formula (1):

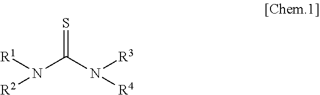

where R¹ to R⁴ each represent one selected from the group consisting of a hydrogen atom, an alkyl group, an aromatic ring group, and a heterocyclic group.

### TECHNICAL FIELD

The present invention relates to a polymerization initiator-containing composition and a dental polymerizable composition.

### BACKGROUND ART

In dental treatment of dental caries (bad tooth), a dental caries portion is removed, and then a prosthesis such as a crown and an inlay may be adhered to a tooth by a dental cement. In this case, there has been known that the surface of the tooth is treated with a dental primer prior to adhering the prosthesis to the tooth, thereby improving adhesiveness of the dental cement to the tooth.

As such a dental primer, there is proposed, for example, a dental primer including a (meth)acrylate having an acid group, a copper compound, a polymerization inhibitor, and water (see, for example, Patent Document 1).

In dental treatment of dental caries (bad tooth), a dental caries portion is removed, and then a cavity of the tooth may be filled with a dental filling material such as a composite resin to restore the tooth. In this case, there has been known that a dental bonding material is applied into the cavity prior to filling the cavity with the dental filling material, thereby improving adhesiveness of the dental filling material to the tooth.

### CITATION LIST

### Patent Document

Patent Document 1: Japanese Unexamined Patent Publication No. 2018-168099

### SUMMARY OF THE INVENTION

### Problem to be Solved by the Invention

In recent years, from the viewpoint of improving the efficiency of dental treatment, a dental material that can be used in both a dental primer and a dental bonding material has been desired. Since, however, the composition of the dental cement and the composition of the dental filling material are different, it is difficult to achieve both adhesion to the dental cement and adhesion to the dental filling material.

For example, the dental primer described in Patent Document 1 can ensure adhesion to the dental cement, but cannot sufficiently ensure adhesion to the dental filling material.

The dental primer and the dental bonding material are required to have excellent discoloration resistance from the viewpoint of aesthetics of the treated tooth.

The present invention provides a polymerization initiator-containing composition and a dental polymerizable composition that can ensure adhesion to both a dental cement and a dental filling material and that can be used in both a dental primer and a dental bonding material, the polymerization initiator-containing composition and the dental polymerizable composition having excellent discoloration resistance.

### Means for Solving the Problem

The present invention [1] includes a polymerization initiator-containing composition including a fourth period transition metal compound including a transition metal atom belonging to the fourth period of the Periodic Table; a tertiary amine compound; and a thiourea compound represented by the following general formula (1) in one agent:

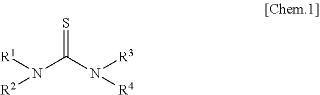

where R¹ to R⁴ each represent one selected from the group consisting of a hydrogen atom, an alkyl group, an aromatic ring group, and a heterocyclic group.

The present invention [2] includes the polymerization initiator-containing composition described in [1], wherein the fourth period transition metal compound includes a copper compound.

The present invention [3] includes the polymerization initiator-containing composition described in [1] or [2], wherein the tertiary amine compound has an aromatic ring.

The present invention [4] includes the polymerization initiator-containing composition described in [3], wherein the tertiary amine compound includes dialkylaminobenzoic ester.

The present invention [5] includes the polymerization initiator-containing composition described in any one of the above-described [1] to [4], wherein the thiourea compound includes trialkylthiourea or tetraalkylthiourea.

The present invention [6] includes a dental polymerizable composition including a polymerization initiator-containing composition described in any one of the above-described [1] to [5]; and a polymerizable monomer.

The present invention [7] includes the dental polymerizable composition described in [6], wherein the polymerizable monomer includes an acidic group-containing polymerizable monomer.

### Effects of the Invention

The polymerization initiator-containing composition of the present invention contains the above-described fourth period transition metal compound and tertiary amine compound in one agent. Therefore, the polymerization initiator-containing composition can impart adhesion to both a dental cement and a dental filler to the dental polymerizable composition.

The polymerization initiator-containing composition further contains the above-described thiourea compound in one agent. Therefore, even though the polymerization initiator-containing composition contains the fourth period transition metal compound and the tertiary amine compound, discoloration resistance of the polymerization initiator-containing composition during storage can be improved.

The dental polymerizable composition of the present invention contains the above-described polymerization initiator-containing composition. Therefore, the dental polymerizable composition can ensure adhesion to a dental filler while ensuring adhesion to a dental cement, and can improve discoloration resistance during storage.

As a result, the polymerization initiator-containing composition and the dental polymerizable composition can be suitably used in both a dental primer and a dental bonding material.

### DESCRIPTION OF THE EMBODIMENTS

The polymerization initiator-containing composition of the present invention is a dental polymerization initiator-containing composition, and can be used in both a dental primer and a dental bonding material. The polymerization initiator-containing composition contains a fourth period transition metal compound, a tertiary amine compound, and a thiourea compound as essential components.

### (1) Fourth Period Transition Metal Compound

The fourth period transition metal compound is a polymerization initiator. The fourth period transition metal compound initiates polymerization (radical polymerization) of a polymerizable monomer to be described later. When the polymerization initiator-containing composition contains the fourth period transition metal compound, adhesion of a dental polymerizable composition to be described later to a dental cement can be ensured.

The fourth period transition metal compound includes a transition metal atom belonging to the fourth period of the Periodic Table (hereinafter referred to as fourth period transition metal atom).

The fourth period transition metal atom is a metal atom belonging to the fourth period of the Periodic Table and belonging to groups 3 to 13 of the Periodic Table. The Periodic Table is in accordance with IUPAC Periodic Table of the Elements (version date 22 Jun. 2007) (the same applies to the following).

Examples of the fourth period transition metal atom specifically include scandium (Sc), titanium (Ti), vanadium (V), chromium (Cr), manganese (Mn), iron (Fe), cobalt (Co), nickel (Ni), copper (Cu), zinc (Zn), and gallium (Ga).

Of the fourth period transition metal atoms, preferably, metal atoms belonging to groups 4 to 12 of the Periodic Table are used, more preferably, metal atoms belonging to groups 5 to 12 of the Periodic Table are used.

In other words, the fourth period transition metal compound preferably includes a titanium compound, a vanadium compound, a chromium compound, a manganese compound, an iron compound, a cobalt compound, a nickel compound, a copper compound, and a zinc compound, more preferably includes a vanadium compound, a chromium compound, a manganese compound, an iron compound, a cobalt compound, a nickel compound, a copper compound, and a zinc compound.

Compounds to be combined with the fourth period transition metal can be classified into inorganic materials and organic materials.

Examples of the inorganic material of the compound to be combined with the fourth period transition metal atom include inorganic salts of the fourth period transition metal atoms and metal halides of the fourth period transition metal atoms.

Examples of the inorganic salt of the fourth period transition metal atom include sulfates and nitrates.

Examples of the metal halide of the fourth period transition metal atom include chlorides and bromides.

Examples of the organic material of the compound to be combined with the fourth period transition metal atom include organic salts of the fourth period transition metal atoms and organic metal complexes of the fourth period transition metal atoms.

Examples of the organic salt of the fourth period transition metal atom include carboxylates. Examples of the carboxylate include acetates.

The organic metal complex of the fourth period transition metal atom has a fourth period transition metal atom as a central metal and a ligand coordinated to the fourth period transition metal atom. Examples of the ligand include diketone ligands, amine ligands, and imine ligands.

The fourth period transition metal compounds can be used alone or in combination of two or more.

The fourth period transition metal compound preferably includes an iron compound and/or a copper compound, more preferably includes a copper inorganic compound and/or a copper organic compound, even more preferably includes at least one selected from the group consisting of a copper inorganic salt, a copper organic salt, and a copper halide, even more preferably includes at least one selected from the group consisting of copper sulfate, copper chloride, and copper acetate, even more preferably includes copper sulfate and/or copper acetate. The fourth period transition metal compound preferably consists of an iron compound and/or a copper compound.

When the fourth period transition metal compound includes an iron compound and/or a copper compound, adhesion of the dental polymerizable composition to be described later to a dental cement can be surely improved. In particular, when the fourth period transition metal compound includes a copper compound, adhesion of the dental polymerizable composition to be described later to a dental cement can be further surely improved.

Content ratio of the fourth period transition metal compound in the dental polymerizable composition to be described later is, for example, 0.001 parts by mass or more, preferably 0.01 parts by mass or more, more preferably 0.02 parts by mass or more, and for example, 5.0 parts by mass or less, preferably 3.0 parts by mass or less, more preferably 1.5 parts by mass or less, even more preferably 1.0 part by mass or less, relative to 100 parts by mass of a total sum of the polymerizable monomer to be described later, the fourth period transition metal compound, the tertiary amine compound, and the thiourea compound.

### (2) Tertiary Amine Compound

The tertiary amine compound is a reducing agent. When the polymerization initiator-containing composition contains the tertiary amine compound, adhesion of the dental polymerizable composition to be described later to a composite resin can be ensured.

Examples of the tertiary amine compound include an aromatic ring-containing tertiary amine compound and an aromatic ring-free tertiary amine compound.

The aromatic ring-containing tertiary amine compound has an aromatic ring and a tertiary amino group.

Examples of the aromatic ring-containing tertiary amine compound include N,N-dialkyltoluidine, N,N-bis(hydroxyalkyl)toluidine, N,N-dialkylaniline, and dialkylaminobenzoic esters.

Examples of the N,N-dialkyltoluidine include N,N-dimethyl-p-toluidine and N,N-diethyl-p-toluidine.

Examples of the N,N-bis(hydroxyalkyl)toluidine include N,N-bis(2-hydroxyethyl)-p-toluidine and N,N-bis(2-hydroxypropyl)-p-toluidine.

Examples of the N,N-dialkylaniline include N,N-dimethylaniline and N,N-diethylaniline.

Examples of the dialkylaminobenzoic ester include methyl 4-(dimethylamino)benzoate, ethyl 4-(dimethylamino)benzoate, 2-butoxyethyl 4-(dimethylamino)benzoate, and isoamyl 4-(dimethylamino)benzoate.

The aromatic ring-free tertiary amine compound does not include an aromatic ring but includes a tertiary amino group.

Examples of the aromatic ring-free tertiary amine compound include N,N-dialykylaminoalkyl (meth)acrylate, trialkylamine, and alkanolamine.

Examples of the N,N-dialykylaminoalkyl (meth)acrylate include N,N-dimethylaminoethyl (meth)acrylate and N,N-diethylaminoethyl (meth)acrylate. Examples of the (meth)acrylate include methacrylate and acrylate (the same applies to the following).

Examples of the trialkylamine include trimethylamine, triethylamine, and tripropylamine.

Examples of the alkanolamine include N-ethyldiethanolamine and triethanolamine.

The tertiary amine compounds can be used alone or in combination of two or more.

The tertiary amine compound preferably includes an aromatic ring-containing tertiary amine compound. In other words, the tertiary amine compound preferably has an aromatic ring. The tertiary amine compound more preferably consists of an aromatic ring-containing tertiary amine compound.

The aromatic ring-containing tertiary amine compound preferably includes N,N-dialkyltoluidine and/or dialkylaminobenzoic ester, more preferably includes dialkylaminobenzoic ester, even more preferably includes one selected from the group consisting of ethyl 4-(dimethylamino)benzoate, 2-butoxyethyl 4-(dimethylamino)benzoate, and isoamyl 4-(dimethylamino)benzoate, even more preferably includes ethyl 4-(dimethylamino)benzoate.

When the tertiary amine compound includes the aromatic ring-containing tertiary amine compound, both adhesion to a dental cement and adhesion to a composite resin can be imparted to the dental polymerizable composition to be described later in a well-balanced manner.

In particular, when the tertiary amine compound includes dialkylaminobenzoic ester, adhesion of the dental polymerizable composition to be described later to a composite resin can be further surely improved.

Content ratio of the tertiary amine compound in the dental polymerizable composition to be described later is, for example, 0.01 parts by mass or more, preferably 0.1 parts by mass or more, more preferably 0.5 parts by mass or more, and for example, 10.0 parts by mass or less, preferably 5.0 parts by mass or less, more preferably 2.0 parts by mass or less, relative to 100 parts by mass of the total sum of the polymerizable monomer to be described later, the fourth period transition metal compound, the tertiary amine compound, and the thiourea compound.

### (3) Thiourea Compound

The thiourea compound is a reducing agent. When the polymerization initiator-containing composition contains the thiourea compound, discoloration resistance of the polymerization initiator-containing composition during storage can be improved.

The thiourea compound is represented by the following general formula (1):

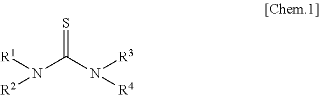

where R¹ to R⁴ each represent one selected from the group consisting of a hydrogen atom, an alkyl group, an aromatic ring group, and a heterocyclic group.

In the general formula (1), the alkyl group represented by R¹ to R⁴ preferably represents an alkyl group having 1 to 10 carbon atoms, more preferably an alkyl group having 1 to 4 carbon atoms, even more preferably a methyl group.

Examples of the heterocyclic group represented by R¹ to R⁴ in the general formula (1) include a phenyl group and a benzyl group.

Examples of the heterocyclic group represented by R¹ to R⁴ in the general formula (1) include a pyridyl group and a pyrazine group.

In the general formula (1), R¹ to R⁴ may be the same as or different from each other.

The thiourea compounds can be used alone or in combination of two or more.

The thiourea compound preferably includes at least one selected from the group consisting of thiourea, alkylthiourea, aromatic ring-containing thiourea, and heterocyclic ring-containing thiourea. The thiourea compound more preferably consists of at least one selected from the group consisting of thiourea, alkylthiourea, aromatic ring-containing thiourea, and heterocyclic ring-containing thiourea.

When the thiourea compound is thiourea, all of R¹ to R⁴ in the general formula (1) represent hydrogen atoms.

Examples of the alkylthiourea include tetraalkylthiourea and trialkylthiourea.

When the thiourea compound is tetraalkylthiourea, all of R¹ to R⁴ in the general formula (1) represent alkyl groups. Examples of the tetraalkylthiourea include tetramethylthiourea and tetraethylthiourea.

When the thiourea compound is trialkylthiourea, in the general formula (1), one of R¹ to R⁴ represents a hydrogen atom and the others represent alkyl groups. Examples of the trialkylthiourea include trimethylthiourea and triethylthiourea.

When the thiourea compound is aromatic ring-containing thiourea, in the general formula (1), at least one of R¹ to R⁴ represents an aromatic ring group and the others represent hydrogen atoms or alkyl groups. Examples of the aromatic ring-containing thiourea include N,N-diphenylthiourea.

When the thiourea compound is heterocyclic ring-containing thiourea, in the general formula (1), at least one of R¹ to R⁴ represents a heterocyclic group and the others represent hydrogen atoms or alkyl groups. Examples of the heterocyclic ring-containing thiourea include (2-pyridyl)thiourea and 1-(pyrazin-2-yl)thiourea.

Of thiourea, alkylthiourea, and heterocyclic ring-containing thiourea, preferably, alkylthiourea is used, more preferably, trialkylthiourea and tetraalkylthiourea are used, even more preferably, tetramethylthiourea is used.

Content ratio of the thiourea compound in the dental polymerizable composition to be described later is, for example, 0.01 parts by mass or more, preferably 0.1 parts by mass or more, more preferably 0.2 parts by mass or more, and for example, 10.0 parts by mass or less, preferably 5.0 parts by mass or less, more preferably 2.0 parts by mass or less, relative to 100 parts by mass of the total sum of the polymerizable monomer to be described later, the fourth period transition metal compound, the tertiary amine compound, and the thiourea compound.

### (4) Optional Components

### (4-1) Solvent

The polymerization initiator-containing composition can further contain a solvent as an optional component.

Examples of the solvent include water and an organic solvent.

Examples of the organic solvent include alcohols and ketones. Examples of the alcohols include ethanol and isopropyl alcohol. Examples of the ketones include acetone.

The solvents can be used alone or in combination of two or more.

The solvent preferably includes water and/or ethanol, more preferably includes water and ethanol. The solvent preferably consists of water and/or ethanol.

Content ratio of the solvent in the polymerization initiator-containing composition is, for example, 0% by mass or more, preferably 5% by mass or more, more preferably 20% by mass or more, even more preferably 30% by mass or more, and for example, 95% by mass or less, preferably 85% by mass or less, more preferably 75% by mass or less, even more preferably 65% by mass or less, particularly preferably 45% by mass or less.

### (4-2) Other Additives

Further, the polymerization initiator-containing composition can contain other additives as optional components at appropriate ratios. Examples of the other additives include photopolymerization initiators, polymerization inhibitors, reducing agents, fillers, and silane coupling agents.

### (5) Operations and Effects

The above-described polymerization initiator-containing composition contains the above-described fourth period transition metal compound and tertiary amine compound in one agent. Therefore, the polymerization initiator-containing composition can impart both adhesion to a dental cement and adhesion to a dental filler to the dental polymerizable composition to be described later.

The polymerization initiator-containing composition also contains the above-described thiourea compound. Therefore, even though the polymerization initiator-containing composition contains the fourth period transition metal compound and the tertiary amine compound, discoloration resistance of the polymerization initiator-containing composition during storage can be improved.

As a result, the polymerization initiator-containing composition can be suitably used in both a dental primer and a dental bonding material.

### (6) Dental Polymerizable Composition

The above-described polymerization initiator-containing composition is suitable for medical polymerizable compositions, in particular, for dental polymerizable compositions. The dental polymerizable composition contains the above-described polymerization initiator-containing composition and a polymerizable monomer.

### (6-1) Polymerizable Monomer

Examples of the polymerizable monomer include acidic group-containing polymerizable monomers and acidic group-free polymerizable monomers.

The acidic group-containing polymerizable monomer has an acidic group and a polymerizable group.

Examples of the acidic group include free acidic groups, and acidic groups forming acid anhydrides.

The acidic group is located, for example, at one end of the molecule of the acidic group-containing polymerizable monomer.

Examples of the free acidic group include a carboxy group, a phosphate group, and a sulfo group.

These acidic groups can form an acid anhydride. An acid anhydride is formed by subjecting two acidic groups to dehydration condensation. Hereinafter, the acidic group that forms the acid anhydride is referred to as an acid anhydride of the acidic group.

Examples of the acid anhydride of the acidic group include an acid anhydride of carboxy groups, an acid anhydride of phosphate groups, and an acid anhydride of sulfo groups.

The acidic group-containing polymerizable monomer may contain the acidic group or the acid anhydride of the acidic group alone, or may use two or more acidic groups and/or acid anhydrides of acidic groups.

The polymerizable group is located, for example, at an end (the other end) on the side opposite to the acidic group in the molecule of the acidic group-containing polymerizable monomer.

Examples of the polymerizable group include ethylenically unsaturated double bonds, specifically, a (meth)acryloyloxy group, a (meth)acryloylamide group, a vinyl group, and a vinyl cyanide group. Of these polymerizable groups, preferably, a (meth)acryloyloxy group is used. Examples of the (meth)acryloyl include methacryloyl and acryloyl (the same applies to the following).

The acidic group-containing polymerizable monomer has a molecular weight of, for example, 70 or more, preferably 200 or more, and for example, 600 or less, preferably 400 or less.

Examples of the acidic group-containing polymerizable monomer include carboxy-type polymerizable monomers, phosphoric acid-type polymerizable monomers, and sulfo-type polymerizable monomers.

The carboxy-type polymerizable monomer has a carboxy group and/or an acid anhydride of carboxy groups.

The phosphoric acid-type polymerizable monomer has a phosphoric acid group and/or an acid anhydride of phosphoric acid groups.

The sulfo-type polymerizable monomer has a sulfo group and/or an acid anhydride with sulfo groups.

The acidic group-containing polymerizable monomers can be used alone or in combination of two or more.

The acidic group-containing polymerizable monomer preferably includes a carboxy-type polymerizable monomer and/or a phosphoric acid-type polymerizable monomer, more preferably, includes a phosphoric acid-type polymerizable monomer. The acidic group-containing polymerizable monomer more preferably consists of a carboxy-type polymerizable monomer and/or a phosphoric acid-type polymerizable monomer.

As the carboxy-type polymerizable monomer, specifically, the carboxyl group-containing polymerizable monomer exemplified in paragraph [0043] of Japanese Unexamined Patent Publication No. 2006-347943 is used, preferably, 4-(meth)acryloyloxyethyl trimellitate anhydride is used, more preferably, 4-methacryloyloxyethyl trimellitate anhydride (hereinafter referred to as 4-META) is used.

As the phosphoric acid-type polymerizable monomer, specifically, the phosphoric acid group-containing polymerizable monomer exemplified in paragraph [0045] of Japanese Unexamined Patent Publication No. 2006-347943 is used, preferably, 10-(meth)acryloyloxydecyl dihydrogen phosphate is used, more preferably, 10-methacryloyloxydecyl dihydrogen phosphate (hereinafter referred to as MDP) is used.

MDP consists of a free phosphoric acid group located at one end of its molecule, a methacryloyloxy group (polymerizable group) located at the other end of its molecule, and a decylene group that bonds a phosphoric acid group and a methacryloyloxy group.

The acidic group-free polymerizable monomer has the above-described polymerizable groups and does not have the above-described acidic groups.

The acidic group-free polymerizable monomer has a molecular weight of, for example, 70 or more, preferably 100 or more, and for example, 1000 or less, preferably 700 or less.

Examples of the acidic group-free polymerizable monomer include hydroxyl group-containing polymerizable monomers and hydroxyl group-free polymerizable monomers.

The hydroxyl group-containing polymerizable monomer does not have the above-described acidic groups and has a hydroxyl group.

Examples of the hydroxyl group-containing polymerizable monomer include hydroxyl group-containing mono(meth)acrylate, hydroxyl group-containing di(meth)acrylate, and hydroxyl group-containing (meth)acrylamide.

The hydroxyl group-containing polymerizable monomers can be used alone or in combination of two or more.

The hydroxyl group-containing polymerizable monomer preferably includes hydroxyl group-containing mono(meth)acrylate. The hydroxyl group-containing polymerizable monomer more preferably consists of hydroxyl group-containing mono(meth)acrylate.

Examples of the hydroxyl group-containing mono(meth)acrylate include 2-hydroxyethyl (meth)acrylate, 3-hydroxypropyl (meth)acrylate, 6-hydroxyhexyl (meth)acrylate, and dihydroxypropyl mono(meth)acrylate.

Of these hydroxyl group-containing mono(meth)acrylates, preferably, 2-hydroxyethyl (meth)acrylate is used, preferably, 2-hydroxyethyl methacrylate (HEMA) is used.

The hydroxyl group-free polymerizable monomer does not have the above-described acidic groups or hydroxyl groups.

Examples of the hydroxyl group-free polymerizable monomer include monofunctional (meth)acrylate, bifunctional (meth)acrylate, and trifunctional (meth)acrylate.

The hydroxyl group-free polymerizable monomers can be used alone or in combination of two or more.

The hydroxyl group-free polymerizable monomer preferably includes bifunctional (meth)acrylate. The hydroxyl group-free polymerizable monomer more preferably consists of bifunctional (meth)acrylate.

As the bifunctional (meth)acrylate, preferably, alkylene di(meth)acrylate, urethane di(meth)acrylate, oxyalkylene di(meth)acrylate, and aromatic ring-containing di(meth)acrylate are used, more preferably, urethane di(meth)acrylate is used.

The urethane di(meth)acrylate has a urethane bond. Examples of the urethane di(meth)acrylate include [2,2,4-trimethylhexamethylenebis(2-carbamoyloxyethyl)]dimethacrylate.

The polymerizable monomers can be used alone or in combination of two or more.

The polymerizable monomer preferably includes an acidic group-containing polymerizable monomer, more preferably includes an acidic group-containing polymerizable monomer and a hydroxyl group-free polymerizable monomer, even more preferably, includes an acidic group-containing polymerizable monomer, a hydroxyl group-containing polymerizable monomer, and a hydroxyl group-free polymerizable monomer.

When the polymerizable monomer contains an acidic group-containing polymerizable monomer, content ratio of the acidic group-containing polymerizable monomer in the polymerizable monomer is, for example, 5% by mass or more, preferably 15% by mass or more, and for example, 100% by mass or less, preferably 80% by mass or less, more preferably 50% by mass or less, even more preferably 30% by mass or less.

When the polymerizable monomer contains a hydroxyl group-free polymerizable monomer, content ratio of the hydroxyl group-free polymerizable monomerin the polymerizable monomer is, for example, 10% by mass or more, preferably 20% by mass or more, more preferably 40% by mass or more, even more preferably 60% by mass or more, and for example, 90% by mass or less, preferably 80% by mass or less.

When the polymerizable monomer contains a hydroxyl group-containing polymerizable monomer, content ratio of the hydroxyl group-free polymerizable monomer in the polymerizable monomer is, for example, 1% by mass or more, preferably 5% by mass or more, and for example, 30% by mass or less, preferably 20% by mass or less.

Further, content ratio of the polymerizable monomer in the dental polymerizable composition is, for example, 0% by mass or more, preferably 50% by mass or more, more preferably 60% by mass or more, and for example, 99.3% by mass or less, preferably 90% by mass or less, more preferably 80% by mass or less.

The polymerizable monomer is in a proportion of, for example, 0 parts by mass or more, preferably 1000 parts by mass or more, more preferably 2000 parts by mass or more, even more preferably 5000 parts by mass or more, and for example, 20000 parts by mass or less, preferably 15000 parts by mass or less, more preferably 10000 parts by mass or less, relative to 100 parts by mass of a total sum of the fourth period transition metal compound, the tertiary amine compound, and the thiourea compound.

Such dental polymerizable composition can ensure adhesion to a dental filler while ensuring adhesion to a dental cement, and can improve discoloration resistance during storage. Therefore, the dental polymerizable composition can be suitably used in both a dental primer and a dental bonding material. That is, the dental polymerizable composition is a dental primer-cum-bonding material.

Specifically, when the dental polymerizable composition is used as a dental primer, for example, the dental polymerizable composition is applied to a preformed abutment tooth. A dental cement is applied to a prosthesis. The dental cement includes, for example, a polymerizable monomer, a filler, a photopolymerization initiator, and a chemical polymerization initiator. The prosthesis is fitted to the abutment tooth so that the dental cement comes into contact with the dental polymerizable composition thus applied to the abutment tooth. Thereafter, the dental cement is cured. Thus, the prosthesis is securely bonded to the abutment tooth.

When the dental polymerizable composition is used as a dental bonding material, the dental polymerizable composition is applied into a cavity of a tooth. Then, the cavity is filled with a composite resin. The composite resin includes, for example, a polymerizable monomer, a tertiary amine compound, a photopolymerization initiator, and a filler. Thereafter, the composite resin is cured. Thus, the composite resin is securely bonded to the tooth.

### EXAMPLE

Hereinafter, the present invention will be described more in detail with reference to Examples and Comparative Examples, but not limited thereto. The specific numerical values in mixing ratio (content ratio), property value, and parameter used in the following description can be replaced with upper limit values (numerical values defined as “or less” or “below”) or lower limit values (numerical values defined as “or more” or “above”) of corresponding numerical values in mixing ratio (content ratio), property value, and parameter described in the above-described “DESCRIPTION OF THE EMBODIMENTS”.

### Examples 1 to 26 and Comparative Examples 1 to 17

According to the formulations in Tables 1 to 3, a solvent, a tertiary amine compound, a fourth period transition metal compound, and a thiourea compound were mixed to prepare a polymerization initiator-containing composition. Then, according to the formulations in Tables 1 to 3, the polymerization initiator-containing composition and a polymerizable monomer were mixed to prepare a dental polymerizable composition.

### Evaluation

### 1. Adhesion Test

### 1.1 Adhesion of Dental Polymerizable Composition to Dental Cement

A stainless steel rod having a diameter of 6 mm was polished with a #400 durable abrasive paper and was then sandblasted. Next, the rod was treated by a Super-Bond (registered trademark) PZ Primer (manufactured by San Medical Co., Ltd.) and then used as a tensile test jig.

Next, enamel of a bovine tooth was polished with a #180 durable abrasive paper so as to be flat. Thereafter, a 150 μm-thick seal having a 4.8 mm-diameter hole was adhered to the enamel of the bovine tooth, and an area to be adhered was specified.

The dental polymerizable composition prepared in each of Examples and Comparative Examples was applied as a dental primer to the surface of the enamel of the bovine tooth exposed from the hole in the seal. A coated film of the dental polymerizable composition was dried by air blow so that the coated film of the dental polymerizable composition had a uniform thickness.

Next, an appropriate amount of a kneaded material of a primer combined type resin cement, PANAVIA (registered trademark) V5 (dental cement, manufactured by Kuraray Noritake Dental Inc.) was applied to an adherend surface that was subjected to a primer treatment with the dental polymerizable composition. Then, the tensile test jig was pressure-welded to the applied resin cement. Thereafter, the resulting product was allowed to stand still for 30 minutes and was then immersed in water at 37° C. overnight. In this manner, a test object was prepared.

Next, a universal tester, Autograph AG-IS (manufactured by Shimadzu Corporation), was used under conditions of a crosshead speed of 2 mm/min to measure tensile bond strength of three test objects. Then, an average value of the three measured values was determined as a measurement result. Then, adhesion to the dental cement when the dental polymerizable composition was used as a dental primer was evaluated by the following criteria. The results are shown in Tables 1 and 2.

Good: The measurement result was 2 MPa or more.
Bad: The measurement result was less than 2 MPa.

### 1.2 Adhesion of Dental Polymerizable Composition to Composite Resin

A stainless steel rod having a diameter of 6 mm was polished with a #400 durable abrasive paper and was then sandblasted. The obtained rod was used as a tensile test jig.

Next, enamel of a bovine tooth was polished with a #180 durable abrasive paper so as to be flat. Thereafter, a 700 μm-thick cardboard having a 4.8 mm-diameter hole was adhered to the enamel of the bovine tooth with a double-sided adhesive tape, and an area to be adhered was specified.

The dental polymerizable composition prepared in each of Examples and Comparative Examples was applied as a dental bonding material to the surface of the enamel of the bovine tooth exposed from the hole in the seal. A coated film of the dental polymerizable composition was dried by air blow so that the coated film of the dental polymerizable composition had a uniform thickness.

Next, the hole in the cardboard was filled with a BULK BASE (registered trademark) HARD (manufactured by San Medical Co., Ltd.) as a composite resin. Subsequently, a polyester film was placed on the filled composite resin. Thereafter, the composite resin was pressure-welded through the polyester film using a flat glass plate. The composite resin was then photocured by a PenCure (registered trademark) 2000 (manufactured by J. MORITA MFG. CORP.)

Next, the polyester film was removed, and using a Super-Bond (registered trademark, manufactured by San Medical Co., Ltd.), the tensile test jig was implanted in the cured composite resin. Thereafter, the resulting product was allowed to stand still for 30 minutes and was then immersed in water at 37° C. overnight. In this manner, a test object was prepared.

Next, a universal tester, Autograph AG-IS (manufactured by Shimadzu Corporation), was used under conditions of a crosshead speed of 2 mm/min to measure tensile bond strength of three test objects. Then, an average value of the three measured values was determined as a measurement result. Then, adhesion to the composite resin when the dental polymerizable composition was used as a dental bonding material was evaluated by the following criteria. The results are shown in Tables 1 and 2.

Good: The measurement result was 2 MPa or more.
Bad: The measurement result was less than 2 MPa.

### <Discoloration Resistance>

The dental polymerizable composition prepared in each of Examples and Comparative Examples was sealed in a glass bottle and left for 24 hours under an environment of 55° C. Thereafter, discoloration of the dental polymerizable composition was visually checked. Then, the discoloration resistance (storage stability) during storage was evaluated by the following criteria. The results are shown in Tables 1 and 2.

Good: Not discolored as compared to before being left under the environment of 55° C.
Bad: Discolored as compared to before being left under the environment of 55° C.

TABLE 1

| | Formulations [parts by mass] | | | | | | | | | | | | | |
|---|---|---|---|---|---|---|---|---|---|---|---|---|---|---|
| | | | | | | | | | | | Fourth period transition | | | |
| | | | | | | | | | | | metal compound | | | |
| | | | | | | | | Tertiary amine compound | | | Copper | | | Iron |
| | | | | | | | | Ethyl 4- | N,N- | | (II) | | | (II) |
| | Polymerizable monomer | | | | | | | (dimethyl | dimethyl- | | sulfate | Copper | Copper | sulfate |
| | | 4- | | | Solvent | | | amino) | p- | Trietha- | penta- | (I) | (II) | hepta- |
| No | MDP | META | UDMA | HEMA | Water | Ethanol | Acetone | benzoate | toluidine | nolamine | hydrate | chloride | acetate | hydrate |
| Ex. 1 | 13.6 | — | 50.0 | 9.1 | 9.1 | 18.2 | — | 0.5 | — | — | 0.2 | — | — | — |
| Ex. 2 | 99.1 | — | — | — | — | — | — | 0.5 | — | — | 0.2 | — | — | — |
| Ex. 3 | 79.1 | — | 20.0 | — | — | — | — | 0.5 | — | — | 0.2 | — | — | — |
| Ex. 4 | 69.1 | — | 20.0 | — | 10.0 | — | — | 0.5 | — | — | 0.2 | — | — | — |
| Ex. 5 | 69.1 | — | 20.0 | — | — | 10.0 | — | 0.5 | — | — | 0.2 | — | — | — |
| Ex. 6 | 59.1 | — | 20.0 | — | 10.0 | 10.0 | — | 0.5 | — | — | 0.2 | — | — | — |
| Ex. 7 | 13.6 | — | 50.0 | 9.1 | 9.1 | 18.2 | — | 0.5 | — | — | 0.2 | — | — | — |
| Ex. 8 | 13.6 | — | 50.0 | 9.1 | 9.1 | 18.2 | — | 0.5 | — | — | 0.2 | — | — | — |
| Ex. 9 | 13.6 | — | 50.0 | 9.1 | 9.1 | 18.2 | — | 0.5 | — | — | 0.2 | — | — | — |
| Ex. 10 | 13.6 | — | 50.0 | 9.1 | 9.1 | 18.2 | — | 0.5 | — | — | — | 0.2 | — | — |
| Ex. 11 | 13.6 | — | 50.0 | 9.1 | 9.1 | 18.2 | — | 0.5 | — | — | — | — | — | 0.2 |

| | Formulations [parts by mass] | | | | | | Evaluation results | | | | |
|---|---|---|---|---|---|---|---|---|---|---|---|
| | Thiourea compound | | | | | | | | | Measurement | |
| | Tetra- | Tri- | | (2- | N,N- | | | Measurement | | result | Adhesion |
| | methyl- | methyl | | pyridyl) | diphenyl- | | Discoloration | result | Adhesion | (Bonding | (Bonding |
| No | thiourea | thiourea | Thiourea | thiourea | thiourea | Total | resistance | (Primer) | (Primer) | material) | material) |
| Ex. 1 | 0.2 | — | — | — | — | 100.9 | Good | 5.3 | Good | 9.4 | Good |
| Ex. 2 | 0.2 | — | — | — | — | 100.0 | Good | 3.2 | Good | 3.1 | Good |
| Ex. 3 | 0.2 | — | — | — | — | 100.0 | Good | 4.1 | Good | 2.9 | Good |
| Ex. 4 | 0.2 | — | — | — | — | 100.0 | Good | 4.8 | Good | 3.7 | Good |
| Ex. 5 | 0.2 | — | — | — | — | 100.0 | Good | 6.4 | Good | 2.7 | Good |
| Ex. 6 | 0.2 | — | — | — | — | 100.0 | Good | 8.0 | Good | 2.9 | Good |
| Ex. 7 | — | 0.2 | — | — | — | 100.9 | Good | 6.7 | Good | 7.0 | Good |
| Ex. 8 | — | — | 0.2 | — | — | 100.9 | Good | 4.6 | Good | 3.9 | Good |
| Ex. 9 | — | — | — | 0.2 | — | 100.9 | Good | 5.4 | Good | 7.3 | Good |
| Ex. 10 | 0.2 | — | — | — | — | 100.9 | Good | 5.6 | Good | 11.0 | Good |
| Ex. 11 | 0.2 | — | — | — | — | 100.9 | Good | 8.7 | Good | 7.1 | Good |

TABLE 2

| | Formulations [parts by mass] | | | | | | | | | | | | | |
|---|---|---|---|---|---|---|---|---|---|---|---|---|---|---|
| | | | | | | | | | | | Fourth period transition | | | |
| | | | | | | | | | | | metal compound | | | |
| | | | | | | | | Tertiary amine compound | | | Copper | | | Iron |
| | | | | | | | | Ethyl 4- | N,N- | | (II) | | | (II) |
| | Polymerizable monomer | | | | | | | (dimethyl | dimethyl- | | sulfate | Copper | Copper | sulfate |
| | | 4- | | | Solvent | | | amino) | p- | Trietha- | penta- | (I) | (II) | hepta- |
| No | MDP | META | UDMA | HEMA | Water | Ethanol | Acetone | benzoate | toluidine | nolamine | hydrate | chloride | acetate | hydrate |
| Ex. 12 | 13.6 | — | 50.0 | 9.1 | 9.1 | 18.2 | — | 0.5 | — | — | 0.2 | — | — | — |
| Ex. 13 | 13.6 | — | 50.0 | 9.1 | 9.1 | 18.2 | — | 0.5 | — | — | 0.2 | — | — | — |
| Ex. 14 | 13.6 | — | 50.0 | 9.1 | 9.1 | 18.2 | — | 0.5 | — | — | 0.2 | — | — | — |
| Ex. 15 | 13.6 | — | 50.0 | 9.1 | 9.1 | 18.2 | — | 0.5 | — | — | 0.2 | — | — | — |
| Ex. 16 | 13.6 | — | 50.0 | 9.1 | 9.1 | 18.2 | — | 0.5 | — | — | 0.5 | — | — | — |
| Ex. 17 | 13.6 | — | 50.0 | 9.1 | 9.1 | 18.2 | — | 0.5 | — | — | 0.5 | — | — | — |
| Ex. 18 | 13.6 | — | 50.0 | 9.1 | 9.1 | 18.2 | — | 0.5 | — | — | 0.5 | — | — | — |
| Ex. 19 | 13.6 | — | 50.0 | 9.1 | 9.1 | 18.2 | — | 0.1 | — | — | 0.2 | — | — | — |
| Ex. 20 | 13.6 | — | 50.0 | 9.1 | 9.1 | 18.2 | — | 1.0 | — | — | 0.2 | — | — | — |
| Ex. 21 | 13.6 | — | 50.0 | 9.1 | 9.1 | 18.2 | — | — | 0.5 | — | 0.2 | — | — | — |
| Ex. 22 | 13.6 | — | 50.0 | 9.1 | 9.1 | 18.2 | — | — | — | 0.5 | 0.2 | — | — | — |
| Ex. 23 | 13.6 | — | 50.0 | 9.1 | 9.1 | 18.2 | — | 0.5 | — | — | — | — | 0.2 | — |
| Ex. 24 | — | 13.6 | 50.0 | 9.1 | 9.1 | — | 18.2 | 0.5 | — | — | — | — | 0.2 | — |
| Ex. 25 | 13.6 | — | 50.0 | 9.1 | 9.1 | 18.2 | — | 0.5 | — | — | — | — | 0.02 | — |
| Ex. 26 | 13.6 | — | 50.0 | 9.1 | 9.1 | 18.2 | — | 2 | — | — | — | — | 0.02 | — |

| | Formulations [parts by mass] | | | | | | Evaluation results | | | | |
|---|---|---|---|---|---|---|---|---|---|---|---|
| | Thiourea compound | | | | | | | | | Measurement | |
| | Tetra- | Tri- | | (2- | N,N- | | | Measurement | | result | Adhesion |
| | methyl- | methyl- | | pyridyl) | diphenyl- | | Discoloration | result | Adhesion | (Bonding | (Bonding |
| No | thiourea | thiourea | Thiourea | thiourea | thiourea | Total | resistance | (Primer) | (Primer) | material) | material) |
| Ex. 12 | 2.0 | — | — | — | — | 102.7 | Good | 5.4 | Good | 6.7 | Good |
| Ex. 13 | 1.0 | — | — | — | — | 101.7 | Good | 5.8 | Good | 6.0 | Good |
| Ex. 14 | 0.4 | — | — | — | — | 101.1 | Good | 6.3 | Good | 3.9 | Good |
| Ex. 15 | 0.1 | — | — | — | — | 100.8 | Good | 8.4 | Good | 7.1 | Good |
| Ex. 16 | 0.05 | — | — | — | — | 101.1 | Good | 5.1 | Good | 7.8 | Good |
| Ex. 17 | 0.1 | — | — | — | — | 101.1 | Good | 7.3 | Good | 11.2 | Good |
| Ex. 18 | 0.25 | — | — | — | — | 101.3 | Good | 8.0 | Good | 8.4 | Good |
| Ex. 19 | 0.2 | — | — | — | — | 100.5 | Good | 8.3 | Good | 8.4 | Good |
| Ex. 20 | 0.2 | — | — | — | — | 101.4 | Good | 8.7 | Good | 6.4 | Good |
| Ex. 21 | 0.2 | — | — | — | — | 100.9 | Good | 4.8 | Good | 8.5 | Good |
| Ex. 22 | 0.2 | — | — | — | — | 100.9 | Good | 4.0 | Good | 4.8 | Good |
| Ex. 23 | 0.2 | — | — | — | — | 100.9 | Good | 7.5 | Good | 5.3 | Good |
| Ex. 24 | 0.2 | — | — | — | — | 100.9 | Good | 2.9 | Good | 3.3 | Good |
| Ex. 25 | — | — | — | — | 0.08 | 100.5 | Good | 12.0 | Good | 6.9 | Good |
| Ex. 26 | 2 | — | — | — | — | 104.0 | Good | 8.2 | Good | 3.1 | Good |

TABLE 3

| | Formulations [parts by mass] | | | | | | | | | | | | | |
|---|---|---|---|---|---|---|---|---|---|---|---|---|---|---|
| | | | | | | | | | | | Fourth period transition | | | |
| | | | | | | | | | | | metal compound | | | |
| | | | | | | | | Tertiary amine compound | | | Copper | | | Iron |
| | | | | | | | | Ethyl 4- | N,N- | | (II) | | | (II) |
| | Polymerizable monomer | | | | | | | (dimethyl | dimethyl- | | sulfate | Copper | Copper | sulfate |
| | | 4- | | | Solvent | | | amino) | p- | Trietha- | penta- | (I) | (II) | hepta- |
| No | MDP | META | UDMA | HEMA | Water | Ethanol | Acetone | benzoate | toluidine | nolamine | hydrate | chloride | acetate | hydrate |
| Comp. | 13.6 | — | 50.0 | 9.1 | 9.1 | 18.2 | — | — | — | — | 0.2 | — | — | — |
| Ex. 1 | | | | | | | | | | | | | | |
| Comp. | 13.6 | — | 50.0 | 9.1 | 9.1 | 18.2 | — | 0.5 | — | — | — | — | — | — |
| Ex. 2 | | | | | | | | | | | | | | |
| Comp. | 99.3 | — | — | — | — | — | — | 0.5 | — | — | 0.2 | — | — | — |
| Ex. 3 | | | | | | | | | | | | | | |
| Comp. | 79.3 | — | 20.0 | — | — | — | — | 0.5 | — | — | 0.2 | — | — | — |
| Ex. 4 | | | | | | | | | | | | | | |
| Comp. | 69.3 | — | 20.0 | — | 10.0 | — | — | 0.5 | — | — | 0.2 | — | — | — |
| Ex. 5 | | | | | | | | | | | | | | |
| Comp. | 69.3 | — | 20.0 | — | — | 10.0 | — | 0.5 | — | — | 0.2 | — | — | — |
| Ex. 6 | | | | | | | | | | | | | | |
| Comp. | 59.3 | — | 20.0 | — | 10.0 | 10.0 | — | 0.5 | — | — | 0.2 | — | — | — |
| Ex. 7 | | | | | | | | | | | | | | |
| Comp. | 13.6 | — | 50.0 | 9.1 | 9.1 | 18.2 | — | 0.5 | — | — | 0.2 | — | — | — |
| Ex. 8 | | | | | | | | | | | | | | |
| Comp. | 13.6 | — | 50.0 | 9.1 | 9.1 | 18.2 | — | 0.5 | — | — | — | 0.2 | — | — |
| Ex. 9 | | | | | | | | | | | | | | |
| Comp. | 13.6 | — | 50.0 | 9.1 | 9.1 | 18.2 | — | 0.5 | — | — | — | — | — | 0.2 |
| Ex. 10 | | | | | | | | | | | | | | |
| Comp. | 13.6 | — | 50.0 | 9.1 | 9.1 | 18.2 | — | 0.5 | — | — | 0.5 | — | — | — |
| Ex. 11 | | | | | | | | | | | | | | |
| Comp. | 13.6 | — | 50.0 | 9.1 | 9.1 | 18.2 | — | 0.5 | — | — | 0.2 | — | — | — |
| Ex. 12 | | | | | | | | | | | | | | |
| Comp. | 13.6 | — | 50.0 | 9.1 | 9.1 | 18.2 | — | — | 0.5 | — | 0.2 | — | — | — |
| Ex. 13 | | | | | | | | | | | | | | |
| Comp. | 13.6 | — | 50.0 | 9.1 | 9.1 | 18.2 | — | — | — | 0.5 | 0.2 | — | — | — |
| Ex. 14 | | | | | | | | | | | | | | |
| Comp. | 13.6 | — | 50.0 | 9.1 | 9.1 | 18.2 | — | 0.5 | — | — | — | — | 0.2 | — |
| Ex. 15 | | | | | | | | | | | | | | |
| Comp. | — | 13.6 | 50.0 | 9.1 | 9.1 | — | 18.2 | 0.5 | — | — | — | — | 0.2 | — |
| Ex. 16 | | | | | | | | | | | | | | |
| Comp. | 13.6 | — | 50.0 | 9.1 | 9.1 | 18.2 | — | 0.5 | — | — | — | — | 0.02 | — |
| Ex. 17 | | | | | | | | | | | | | | |

| | Formulations [parts by mass] | | | | | | Evaluation results | | | | |
|---|---|---|---|---|---|---|---|---|---|---|---|
| | Thiourea compound | | | | | | | | | Measurement | |
| | Tetra- | Tri- | | (2- | N,N- | | | Measurement | | result | Adhesion |
| | methyl- | methyl- | | pyridyl) | diphenyl- | | Discoloration | result | Adhesion | (Bonding | (Bonding |
| No | thiourea | thiourea | Thiourea | thiourea | thiourea | Total | resistance | (Primer) | (Primer) | material) | material) |
| Comp. | 0.2 | — | — | — | — | 100.4 | Good | 9.7 | Good | 1.0 | Bad |
| Ex. 1 | | | | | | | | | | | |
| Comp. | 0.2 | — | — | — | — | 100.7 | Good | 1.7 | Bad | 10.1 | Good |
| Ex. 2 | | | | | | | | | | | |
| Comp. | — | — | — | — | — | 100.0 | Bad | 3.0 | Good | 3.5 | Good |
| Ex. 3 | | | | | | | | | | | |
| Comp. | — | — | — | — | — | 100.0 | Bad | 2.9 | Good | 4.7 | Good |
| Ex. 4 | | | | | | | | | | | |
| Comp. | — | — | — | — | — | 100.0 | Bad | 2.6 | Good | 2.5 | Good |
| Ex. 5 | | | | | | | | | | | |
| Comp. | — | — | — | — | — | 100.0 | Bad | 4.6 | Good | 2.7 | Good |
| Ex. 6 | | | | | | | | | | | |
| Comp. | — | — | — | — | — | 100.0 | Bad | 9.0 | Good | 5.3 | Good |
| Ex. 7 | | | | | | | | | | | |
| Comp. | — | — | — | — | — | 100.7 | Bad | 8.9 | Good | 6.9 | Good |
| Ex. 8 | | | | | | | | | | | |
| Comp. | — | — | — | — | — | 100.7 | Bad | 5.4 | Good | 11.1 | Good |
| Ex. 9 | | | | | | | | | | | |
| Comp. | — | — | — | — | — | 100.7 | Bad | 6.0 | Good | 5.7 | Good |
| Ex. 10 | | | | | | | | | | | |
| Comp. | — | — | — | — | — | 101.0 | Bad | 7.2 | Good | 8.8 | Good |
| Ex. 11 | | | | | | | | | | | |
| Comp. | — | — | — | — | — | 100.7 | Bad | 7.5 | Good | 8.8 | Good |
| Ex. 12 | | | | | | | | | | | |
| Comp. | — | — | — | — | — | 100.7 | Bad | 7.0 | Good | 8.1 | Good |
| Ex. 13 | | | | | | | | | | | |
| Comp. | — | — | — | — | — | 100.7 | Bad | 5.1 | Good | 5.1 | Good |
| Ex. 14 | | | | | | | | | | | |
| Comp. | — | — | — | — | — | 100.7 | Bad | 4.2 | Good | 4.8 | Good |
| Ex. 15 | | | | | | | | | | | |
| Comp. | — | — | — | — | — | 100.7 | Bad | 2.7 | Good | 4.4 | Good |
| Ex. 16 | | | | | | | | | | | |
| Comp. | — | — | — | — | — | 100.5 | Bad | 5.4 | Good | 11.0 | Good |
| Ex. 17 | | | | | | | | | | | |

The details of the abbreviations in Tables 1 to 3 are given in the following.
MDP: 10-methacryloyloxydecyl dihydrogen phosphate
4-META: 4-methacryloyloxyethyl trimellitate anhydride
UDM: urethane dimethacrylate
HEMA: 2-hydroxyethyl methacrylate

While the illustrative embodiments of the present invention are provided in the above description, such is for illustrative purpose only and it is not to be construed restrictively. Modification and variation of the present invention that will be obvious to those skilled in the art is to be covered by the following claims.

### INDUSTRIAL APPLICABILITY

The polymerization initiator-containing composition and dental polymerizable composition according to the present invention are used for dental treatment.

1. A polymerization initiator-containing composition comprising:
a fourth period transition metal compound comprising a transition metal atom belonging to the fourth period of the Periodic Table;
a tertiary amine compound; and
a thiourea compound represented by the following general formula (1) in one agent: 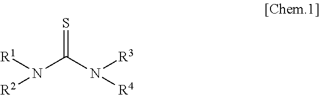
where R¹ to R⁴ each represent one selected from the group consisting of a hydrogen atom, an alkyl group, an aromatic ring group, and a heterocyclic group.

2. The polymerization initiator-containing composition according to claim 1, wherein the fourth period transition metal compound includes a copper compound.

3. The polymerization initiator-containing composition according to claim 1, wherein the tertiary amine compound has an aromatic ring.

4. The polymerization initiator-containing composition according to claim 3, wherein the tertiary amine compound includes dialkylaminobenzoic ester.

5. The polymerization initiator-containing composition according to claim 1, wherein the thiourea compound includes trialkylthiourea or tetraalkylthiourea.

6. A dental polymerizable composition comprising:
a polymerization initiator-containing composition according to claim 1; and
a polymerizable monomer.

7. The dental polymerizable composition according to claim 6, wherein the polymerizable monomer includes an acidic group-containing polymerizable monomer.
